(19) United States
(12) Patent Application Publication
(10) Pub. No.: US 2022/0037029 A1
(43) Pub. Date: Feb. 3, 2022

(54) INFECTIOUS DISEASE HEAT MAP AND PREDICTION MODEL GENERATION

(71) Applicant: T-Mobile USA, Inc., Bellevue, WA (US)

(72) Inventors: ChihChin Wu, Bellevue, WA (US); Boua Lor, Renton, WA (US); Jie Hui, Mercer Island, WA (US); Yasmin Karimli, Kirkland, WA (US)

(21) Appl. No.: 17/338,503

(22) Filed: Jun. 3, 2021

Related U.S. Application Data
(60) Provisional application No. 63/058,404, filed on Jul. 29, 2020.

(51) Int. Cl. G16H 50/30; G16H 50/80; G16H 50/20; A61B 5/11

(52) U.S. Cl. CPC G16H 50/30; A61B 5/11; G16H 50/20; G16H 50/80

(57) ABSTRACT

Described herein are techniques for calculating a likelihood of infection for one or more individuals using heat maps. Such techniques may comprise receiving, in relation to an infectious disease, an indication of an infected individual, determining a first movement data for the infected individual, and updating density information included within one or more heat maps based on the first movement data for the infected individual. The techniques may further comprise determining a second movement data for at least one second individual and calculating a likelihood of infection for the at least one second individual based on the second movement data and the updated one or more heat maps.

### CROSS REFERENCES TO RELATED APPLICATIONS

This application claims the benefit of U.S. Provisional patent application No. 63/058,404, filed on Jul. 29, 2020, which is incorporated herein by reference in its entirety.

### BACKGROUND

As recent events have shown, diseases can spread throughout a population very rapidly. This is exacerbated by the fact that some diseases can become communicable even before symptoms start to appear. During an outbreak of a disease, people that are infected but are as of yet asymptomatic may not know to avoid others and may wind up mingling with friends and family and worsening the outbreak.

### SUMMARY

Techniques are provided herein for generating heat maps associated with an infectious disease. In some embodiments, individuals are identified as having been infected with an infectious disease. The individuals' movement data are then retrieved for some period of time in the past. This movement data may then be used to generate one or more heat maps. Such heat maps may include an indication of a density of infected individuals within a region. In some embodiments, the generated heat maps may vary to reflect the density of infected individuals by location and time.

Embodiments of the disclosure provide several advantages over conventional systems. For example, embodiments of the system described herein enable users to be provided with an indication of the users' likelihood of being infected with an infectious disease without requiring any short-range communication between the users' mobile devices. This provides the ability to perform contact tracing while maintaining a high degree of anonymity. Additionally, the system described herein enables heat maps to be generated that have a higher degree of accuracy than would otherwise be capable of being generated. Heat maps generated using the disclosed system are capable of depicting a density of infected individuals that is accurate down to the feet/meter level.

In one embodiment, a method is disclosed as being performed by a service provider platform, the method comprising receiving, in relation to an infectious disease, an indication of an infected individual, determining a first movement data for the infected individual, updating density information included within one or more heat maps based on the first movement data for the infected individual, determining a second movement data for at least one second individual, and calculating a likelihood of infection for the at least one second individual based on the second movement data and the updated one or more heat maps.

An embodiment is directed to a computing device comprising: a processor; and a memory including instructions that, when executed with the processor, cause the computing device to receive, in relation to an infectious disease, an indication of an infected individual, determine a first movement data for the infected individual, update density information included within one or more heat maps based on the first movement data for the infected individual, determine a second movement data for at least one second individual, and calculate a likelihood of infection for the at least one second individual based on the second movement data and the updated one or more heat maps.

An embodiment is directed to a non-transitory computer-readable media collectively storing computer-executable instructions that upon execution cause one or more computing devices to receive, in relation to an infectious disease, an indication of an infected individual, determine a first movement data for the infected individual, update density information included within one or more heat maps based on the first movement data for the infected individual, determine a second movement data for at least one second individual, and calculate a likelihood of infection for the at least one second individual based on the second movement data and the updated one or more heat maps.

The foregoing, together with other features and embodiments, will become more apparent upon referring to the following specification, claims, and accompanying drawings.

### BRIEF DESCRIPTION OF THE DRAWINGS

The detailed description is described with reference to the accompanying figures, in which the left-most digit(s) of a reference number identifies the figure in which the reference number first appears. The use of the same reference numbers in different figures indicates similar or identical items.

FIG. 1 illustrates an example architecture of a system for predicting a likelihood of infection by generating heat maps and prediction models for infectious diseases in accordance with embodiments;

FIG. 2 is a block diagram showing various components of a computing system architecture that supports generation of heat maps for infectious diseases as well as providing of infection notifications in accordance with embodiments;

FIG. 3 depicts a flow diagram illustrating an exemplary process for generating and using heat maps for infectious diseases in accordance with embodiments;

FIG. 4 depicts

FIG. 5 depicts a number of example heat maps for infectious diseases that may be generated in accordance with embodiments;

FIG. 6 depicts an exemplary GUI in which a user may be presented with vital sign information in accordance with some embodiments;

FIG. 7 depicts an exemplary GUI in which a user may be presented with a notification that he or she may be entering an infected hotspot in accordance with the embodiments described herein;

FIG. 8 depicts an exemplary GUI in which a user may be presented with a notification that he or she may have been exposed to an infectious disease in accordance with the embodiments described herein; and

FIG. 9 depicts a block diagram showing an example process flow for generating one or more heat maps for infectious diseases in accordance with some embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

The present disclosure relates to techniques for generating heat maps and prediction models from individual and aggregated user data. In various embodiments, a user device provides location data to a service provider. The user device may also provide data related to a user's vital signs or other health-related data to the service provider. Vital sign data may be obtained by the user device via an external sensor device in communication with the user device. Based on the provided vital sign data, the service provider or a third-party entity (e.g., hospitals, clinics, laboratories, etc.) working with the service provider may make a determination regarding a status of the user. Such a status may relate to whether the user is currently afflicted with a disease or other ailment. If the service provider determines that a user is potentially a carrier of an infectious disease, the service provider may retrieve historical location data for that user indicating the user's position at various historical points in time.

In some embodiments, historical location data for users determined to be likely infected with an infectious disease may be aggregated to generate a heat map and/or prediction model by the service provider. Such a heat map or prediction model may then be used to provide notifications to other parties. For example, a heat map associated with a current time may be used to notify individuals that are entering into proximity of a highly-infected area that the area is highly-infected. In another example, a heat map may be used to identify disease hotspots so that those hotspots can be treated by emergency personnel. In yet another example, a heat map may be used to balance and assign areas to emergency services (e.g., hospitals) so that they do not become overloaded with cases. In yet another example, heat maps associated with past points in time may be used to identify users that have come in contact with an infected individual in order to provide contact tracing as well as to notify the potentially-infected user.

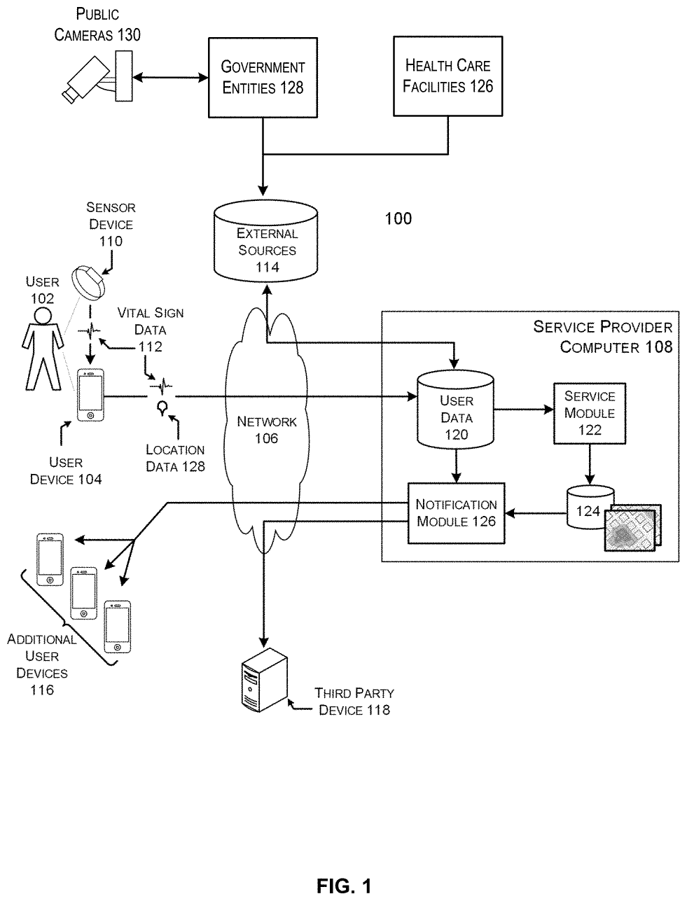

FIG. 1 illustrates an example architecture of a system for predicting a likelihood of infection by generating heat maps and prediction models in accordance with embodiments. As will be appreciated, although a single type of environment is used for purposes of explanation, different environments may be used, as appropriate, to implement various embodiments of the illustrative system. FIG. 1 depicts an illustrative system 100 that includes a user 102 having at least one user device 104 operable to send and receive data over a network 106 to a service provider computer 108. In some embodiments, the user 102 may be in possession of a sensor device 110 capable of collecting and relaying vital sign data 112 to the user device 104. In some embodiments, data may be obtained by the service provider computer 108 from one or more external data sources 114. Additionally, the service provider computer 108 may be configured to provide notifications to one or more additional user devices 116 and/or one or more third party computing device 118.

The user device 104 can include any suitable electronic device operable to send and receive requests, messages or information over an appropriate network 106 and convey information back to a user of the device 104. Examples of such user devices include personal computers, cell phones, handheld messaging devices, laptop computers, set-top boxes, personal data assistants, electronic book readers and the like. In some embodiments, the user 102 may use one or more sensor devices 108 in addition to the user device 104.

The network 106 can include any appropriate network, including an intranet, the Internet, a cellular network, a local area network or any other such network or combination thereof. Components used for such a system can depend at least in part upon the type of network and/or environment selected. Protocols and components for communicating via such a network may be known to one skilled in the art and will not be discussed herein in detail. Communication over the network can be enabled by wired or wireless connections and combinations thereof.

The service provider computer 108 can include any computing device configured to perform at least a portion of the operations described herein. Service provider computer 108 may be composed of one or more general purpose computers, specialized server computers (including, by way of example, PC (personal computer) servers, UNIX™ servers, mid-range servers, mainframe computers, rack-mounted servers, etc.), server farms, server clusters, or any other appropriate arrangement and/or combination. Service provider computer 108 can include one or more virtual machines running virtual operating systems, or other computing architectures involving virtualization such as one or more flexible pools of logical storage devices that can be virtualized to maintain virtual storage devices for the computer.

In accordance with at least some embodiments, the service provider computer 108 may maintain a number of components, including user data 120 that includes a number of details related to one or more users, a service module 122 capable of aggregating user data to generate heat maps and/or predictive models, a data store of location data 124 (e.g., heat maps), and a notification module 126 capable of utilizing the location data 124 to provide notifications to additional user devices 116 and/or at least one third party device 118.

User data 120 may include multiple data stores and may store at least location data 128 received from a plurality of user devices (e.g., user devices 104 and 116). Location data 128 may be obtained by a user device using a global positioning system (GPS) installed within the user device 104. In some cases, location data 128 may be obtained from a user device on a periodic basis (e.g., once every five minutes, etc.) and stored in association with a user. In some embodiments, the user data 120 may also store data related to a status of the user. For example, the service provider computer 108 may receive vital sign data 112 related to the user 102 from the user device 104. Based on the received vital sign data 112, the service provider computer (e.g., the service module 122) may determine that the user is likely currently sick. For example, the service provider computer 108 may determine that the user currently has an elevated body temperature over historic body temperatures for that user, indicating a potential fever. The user data 120 may store historic vital sign data for a number of users as well as a potential status (e.g., potentially-infected, healthy, etc.).

The service module 122 may be configured to identify potentially infected individuals and aggregate user data for those individuals into a heat map and/or predictive model. In some embodiments, the service module 122 may use one or more machine learning techniques to identify potentially infected individuals based on current vital sign data as compared to historical vital sign for each individual. In some embodiments, the service provider computer 108 may be in communication with one or more external data sources 114 that provide infection-related data. For example, the one or more external data sources 114 may include a testing data repository maintained by a disease testing center. The testing repository may provide an indication of a user's current status (e.g., infected, healthy, etc.).

Upon identifying a number of potentially-infected individuals, the service module 122 may retrieve historical location data for each of those individuals. The service module 122 may then aggregate that location data to generate one or more heat maps, which are then stored in location data 124. A heat map, for the purposes of this disclosure, may include any indication of a value of degree of density with respect to a location. A heat map may be associated with a particular point in time. Areas associated with high value (e.g., density) are referred to as hotspots. For example, the system may identify a potential hotspot as an array area that contains more than 10 infected individuals per 100 square meters of area. In some embodiments, a heat map may be a representation of data in the form of a map or diagram in which data values are represented as colors. However, while such a representation may be useful to provide illustration to a human user, some embodiments of a heat map may not include a map at all. In accordance with embodiments herein, a heat map as generated by the service module may indicate a value representing a likelihood of infection with respect to location and time. The service module 122 may update one or more heat maps in the location data 124 as new data is received (e.g., as new infected individuals are identified).

A prediction model, for the purposes of this disclosure, may be any indication of an event or situation that is likely to occur. For example, the service provider, upon generating a number of heat maps, may predict a likely migration of hotspots within the heat maps. For example, the service provider may extrapolate a future trajectory of a hotspot based on a velocity (e.g., speed and direction) of a hotspot within the generated heat maps. Additionally, the service provider may generate a predictive model based on trajectories of likely-infected users. In some embodiments, a predictive model may be generated by training a machine-learning algorithm on provided infection data.

The notification module 126 may be configured to assess the location data 124 and generate a notification to one or more entities based on that assessment. In some embodiments, the notification module 126 may provide one or more generated heat maps to a third-party device 118, such as an emergency service provider (e.g., a hospital). This enables the third-party device 118 to plan for potential cases that it may receive. Additionally, a heat map may be used to identify boundaries for emergency service providers. For example, a number of emergency service providers may set geographical boundaries for servicing based on the heat map such that no particular emergency service provider is overloaded. In some embodiments, the notification module 126 may detect, based on location data for a number of additional user devices 116, that one or more of those additional user devices 116 have entered within, or are about to enter into, a determined infected hotspot.

The illustrative system includes a service provider computer 108. However, it should be understood that a service provider computer 108 can be several computers, layers, or other elements, processes or components, which may be chained or otherwise configured, which can interact to perform tasks such as obtaining data from an appropriate data store. As used herein the term “data store” refers to any device or combination of devices capable of storing, accessing and retrieving data, which may include any combination and number of data servers, databases, data storage devices and data storage media, in any standard, distributed or clustered environment. The service provider computer can include any appropriate hardware and software for integrating with the data store as needed to execute aspects of one or more applications for the user device, handling a majority of the data access and business logic for an application in stalled upon (and executed from) the user device. The service provider computer provides access control services in cooperation with the data store and is able to generate content such as text, graphics, audio and/or video to be transferred to the user device, which may be served to the user in the form of HyperText Markup Language (“HTML”), Extensible Markup Language (“XML”) or another appropriate structured language in this example.

A sensor device 110 may include any device capable of obtaining information about a user and conveying that information to a user device 104. A sensor device 110 may include a number of sensors capable of collecting such information. For example, a sensor device 110 may include a wearable fitness band capable of obtaining at least a user's heart rate and temperature and conveying that heart rate and temperature to the user device 104 via a communication channel (e.g., a Bluetooth™ connection). In another embodiment, the sensor device 110 may be an external sensor that is configured to be connected to the user device 104 (e.g., via a USB connection) and which can collect information from the user.

The system may include a user device that may access the network through an access network via a wireless connection known as the air interface. The air interface may include cellular division multiple access (CDMA), time division multiple access (TDMA), frequency division (FDMA) and future iterations of cellular division, time division and frequency division wireless communications techniques. Examples include orthogonal frequency division multiple access techniques used in current versions of the air interface.

The network side of the air interface terminates with an antenna on a base station which operates as a local smart router. Base stations then access a core network over wired connection known as backhaul. Backhaul is often comprised of fiber optic communications cables. Because multiple antennas and base stations act as collectors funneling user equipment signals to the core network, i.e., providing access to the core network, this portion of the network is known as the access network.

The core network is the portion of the network where routing, billing, policy implementation and other communications services are implemented. Routing may be performed by core network servers such as a Serving GPRS Serving Node (SGSN) and a Gateway GPRS Serving Node (GGSN) for 3G and by Proxy-Call State Control Function (P-CSCF), Serving-Call State Call Function (S-CSCF) and Interrogating-Call State Control Function (I-CSCF) functions for IMS. Service application servers, also known as enterprise information technology (EIT) servers implement application servers. Services are sometimes accessed via the Internet. A gateway provides an interface of the core network to the Internet.

As noted above, the system may receive data from a number of external sources 114. In some embodiments, such external sources may include databases maintained by health care facilities 126 such as hospitals or clinics. In some embodiments, such external sources may include databases maintained by government entities 128 or other agencies responsible for oversight of a region. In some embodiments, one or more such government entities may operate a number of public cameras 130 (e.g., traffic cameras, etc.) that record image data that depicts people in public places. In some cases, a user may be identified within such image data using one or more object recognition techniques. In at least some of these embodiments, such image data may be provided to the system to be used for location tracking/contact tracing of users.

For clarity, a certain number of components are shown in FIG. 1. It is understood, however, that embodiments of the disclosure may include more than one of each component. In addition, some embodiments of the disclosure may include fewer than or greater than all of the components shown in FIG. 1. In addition, the components in FIG. 1 may communicate via any suitable communication medium (including the Internet), using any suitable communication protocol.

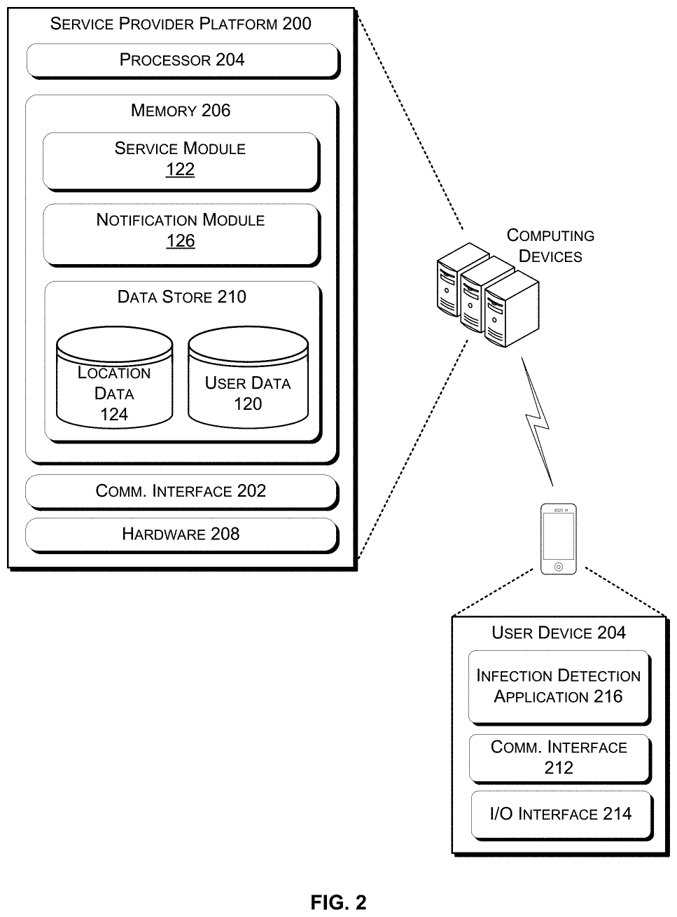

FIG. 2 is a block diagram showing various components of a computing system architecture that supports generation of heat maps as well as providing of infection notifications in accordance with embodiments. The system architecture may include a service provider platform 200 that comprises one or more computing devices. The service provider platform 200 may be in communication with one or more user devices 204.

The service provider platform 200 can include any computing device configured to perform at least a portion of the operations described herein. The service provider platform 200 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. The service provider platform 200 can include one or more virtual machines running virtual operating systems, or other computing architectures involving virtualization such as one or more flexible pools of logical storage devices that can be virtualized to maintain virtual storage devices for the computer. For example, the service provider platform 200 may include virtual computing devices in the form of virtual machines or software containers that are hosted in a cloud.

The service provider platform 200 may include a communication interface 202, one or more processors 204, memory 206, and hardware 208. The communication interface 202 may include wireless and/or wired communication components that enable the service provider platform 200 to transmit data to and receive data from other networked devices. The hardware 208 may include additional user interface, data communication, or data storage hardware. For example, the user interfaces may include a data output device (e.g., visual display, audio speakers), and one or more data input devices. The data input devices may include, but are not limited to, combinations of one or more of keypads, keyboards, mouse devices, touch screens that accept gestures, microphones, voice or speech recognition devices, and any other suitable devices.

The memory 206 may be implemented using computer-readable media, such as computer storage media. Computer-readable media includes, at least, two types of computer-readable media, namely computer storage media and communications media. Computer storage media includes volatile and non-volatile, removable and non-removable media implemented in any method or technology for storage of information such as computer-readable instructions, data structures, program modules, or other data. Computer storage media includes, but is not limited to, RAM, DRAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device. In contrast, communication media may embody computer-readable instructions, data structures, program modules, or other data in a modulated data signal, such as a carrier wave, or other transmission mechanisms.

The one or more processors 204 and the memory 206 of the service provider platform 200 may implement functionality that includes one or more software modules and data stores. Such software modules may include routines, program instructions, objects, and/or data structures that are executed by the processors 204 to perform particular tasks or implement particular data types. More particularly, the memory 206 may include at least a module that is configured to detect potential infections and aggregate user data to generate heat maps and/or predictive models (e.g., service module 122) and a module capable of utilizing the location data 124 to provide notifications to additional user devices (notification module 126). Additionally, the memory 206 may include a data store 210 that includes a number of database tables. For example, the data store 210 may include a database of historic infection data by location (e.g., location data 124) as well as a database of information specific to one or more users (e.g., user data 120).

The service module 122 may be configured to, in conjunction with the processor 204, generate a heat map that indicates a density of infected individuals (either known or suspected) by location and time. In some embodiments, the service module 122 identifies infected individuals. In some cases, infected individuals are identified using self-reporting. In other words, a user may provide an indication to the service provider platform (e.g., via an infection detection application installed on his or her user device) that he or she is infected. In some cases, an infected individual may be identified by one or more external sources. For example, a hospital or other health care organization may provide an indication of one or more people known to be infected. In some embodiments, the service module 122 may identify an infected individual based on determining (using biometric data associated with that individual) that the likelihood of the user being infected is greater than a threshold value. For example, as described elsewhere, the service provider platform may receive biometric data (e.g., vital sign data 112) from a sensor device in communication with the user's user device. In this example, the service module may determine a likelihood that the user is infected based on a variance between the received biometric data values and expected biometric data value thresholds for that user. In some embodiments, expected biometric data for a user may be comprised of a baseline biometric data generated from historic biometric data values for that user. In some embodiments, expected biometric data value thresholds may comprise a set of default biometric data value thresholds that represent an average of each respective data values. In some embodiments, expected biometric data value thresholds may comprise a set of default biometric data value thresholds based on race, gender, age, body mass index, etc. In some embodiments, the service module 122 may query a database of biometric data to obtain a set of expected biometric data value thresholds.

In some embodiments, a determination as to likelihood of infection may be made based on symptoms associated with the infectious disease. For example, if one symptom associated with the infectious disease is a fever, then the service module 122 may determine associate a higher probability that the user is infected with a high body temperature indicated in the biometric data for that user. For example, the service module may determine that any user having a body temperature greater than 100 degrees is likely running a fever and may assign a higher probability that the user is infected.

Upon identifying one or more infected (or likely infected) individuals, the service module may be further configured to aggregate user movement information for the one or more infected individuals into a heat map. To do this, the service module may identify a user's movements throughout a period of time from user data 120. Such movements may be represented by a location of the user at a point in time. In some embodiments, the period of time may be representative of an incubation period for the infectious disease. For example, if an infectious disease typically takes one week from infection to manifest symptoms, then the user's movements may be determined for the last week or week and a half. In some embodiments, upon newly identifying an infected individual, that individual's movement data is used to supplement one or more heat maps within which the user has traveled during the period of time. To do this, the service module may adjust a density of infected individuals of the heat map to account for the infected individual's presence at a particular location and time based on the movements of the infected individual. In this way, historic heat maps may be updated as new cases of an infectious disease are discovered.

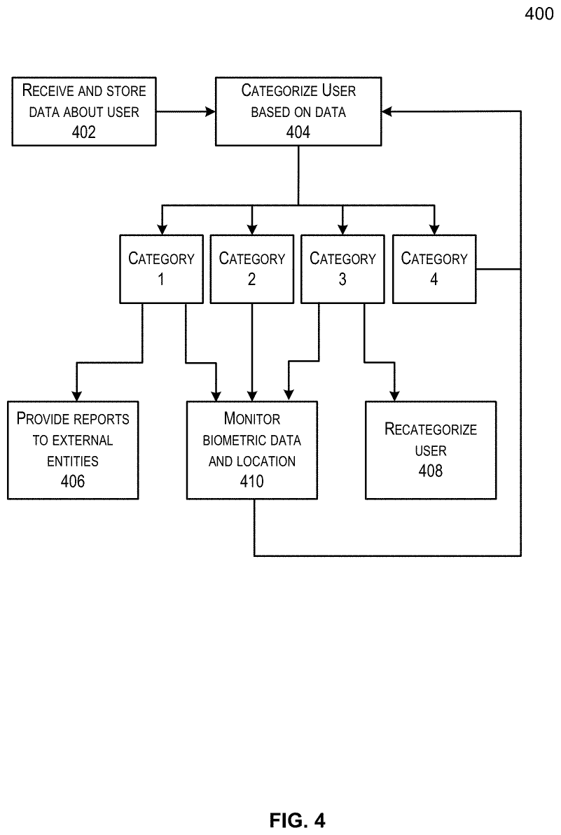

It should be noted that a heat map for a particular region or area may vary based on time. For example, a heat map may indicate a first density of infected individuals at a particular location at a first point in time and a second density of infected individuals at the particular location at a second point in time. This is described in greater detail in FIG. 4 below.

The notification module 126 may be configured to, in conjunction with the processor 204, determine a likelihood that a user has (or is about to) come into contact with an infectious disease and provide a notification to that user in order to stop the spread of the infectious disease. In some embodiments, this may comprise identifying locations associated with a user's movements and determining, using one or more heat maps corresponding to the identified locations (and the time at which the user was determined to be at those locations), a likelihood of the user's exposure to the infectious disease. In some embodiments, such a likelihood of the user's exposure to the infectious disease may be determined based on a density of infected individuals determined to be in proximity to the identified locations. In some cases, that probability may represent an aggregate value calculated based on the user's proximity to infected individuals throughout a preconfigured period of time.

It should be noted that the notification module 126 may update the likelihood of the user's exposure to an infectious disease dynamically (e.g., as new information is received) or periodically (e.g., each minute, hour, day, etc.). For example, when a heat map is updated to include locations and corresponding times associated with known infected individuals, the user's likelihood of exposure to the infectious disease may be updated. In some embodiments, if the user's likelihood of infection rises above a threshold likelihood value, the notification module 126 may be configured to provide a notification to the user. In such embodiments, the notification module 126 may transmit the notification to a user device 204 associated with the user and may be presented via a graphical user interface associated with an infection detection application 216 installed upon, and executed from, the user device 204.

In some embodiments, the service provider platform may include location data 124 that includes a database of historic infection data by location. In some embodiments, such historic infection data may be stored as a heat map or other indicator of a density and/or number of infected individuals by location/area. Such a heat map may be generated using historic user location information obtained from user data 120 with respect to users determined to be infected.

As noted elsewhere, the service provider platform 200 may be in communication with a number of user devices 204. Such a user device 204 may be any electronic device capable of interacting with the service provider platform 200 as described herein and capable of presenting information (e.g., notifications) to a user. User devices 204 may be examples of user devices 104 and/or additional user devices 116 as described with respect to FIG. 1 above. The user device 204 may include a processor and a computer readable memory as well as a communication interface 212 and I/O interface 214. The computer readable memory of the user device 204 may include an infection detection application 216 that enables detection and notification of a potential infection. Additionally, the infection detection application 216 may enable (e.g., via a graphical user interface) interaction between a user of the user device and the service provider platform. For example, execution of the infection detection application 216 on the user device 204 may cause a communication session to be established between the user device and the service provider platform 200. In this example, the service provider platform may provide information to the user device via the communication session that is then presented on the user device. In some embodiments, the infection detection application is configured to interact with the service provider platform using an application programming interface (API) associated with the service provider platform.

The user device 202 may also be equipped with various hardware. Such hardware may include additional user interface, data communication, data storage hardware, or sensors (e.g., a camera, a gyroscope, an accelerometer, a compass, etc.). For example, the user interfaces may include a data output device (e.g., visual display, audio speakers), and one or more data input devices. The data input devices may include but are not limited to, combinations of one or more of keypads, keyboards, mouse devices, touch screens that accept gestures, microphones, voice or speech recognition devices, and any other suitable devices.

The communication interface 212 may potentially work in concert with the I/O interface 214 and may be a network interface card supporting Ethernet and/or Wi-Fi and/or any number of other physical and/or datalink protocols. Generally, a supported network will include a communication network. Accordingly, the network interface 212 will include a cellular radio and radio access software layer. The I/O interface 214 may be any controller card, such as a universal asynchronous receiver/transmitter (UART) used in conjunction with a standard I/O interface protocol such as RS-232 and/or Universal Serial Bus (USB).

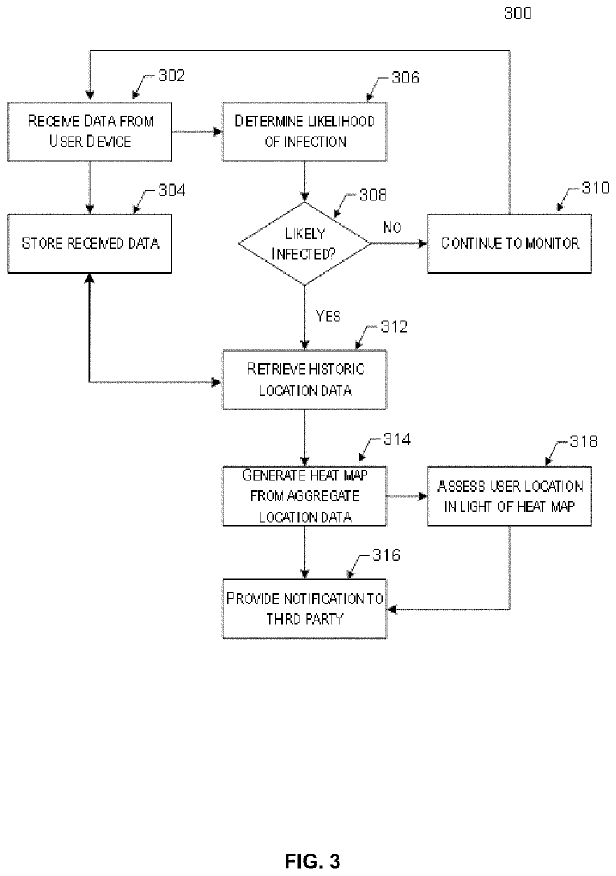

FIG. 3 depicts a flow diagram illustrating an exemplary process for generating and using heat maps in accordance with embodiments. The process 300 is illustrated as a logical flow diagram, each operation of which represents a sequence of operations that can be implemented in hardware, computer instructions, or a combination thereof. In the context of computer instructions, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be omitted or combined in any order and/or in parallel to implement this process and any other processes described herein.

Some or all of the exemplary process 300 (or any other processes described herein, or variations and/or combinations thereof) may be performed under the control of one or more computer systems configured with executable instructions and may be implemented as code (e.g., executable instructions, one or more computer programs or one or more applications). In accordance with at least one embodiment, the process 300 of FIG. 3 may be performed by one or more computing devices as shown in FIG. 1. For example, the process 300 may be performed by a service provider computer 108 as described with respect to FIG. 1. The code may be stored on a computer-readable storage medium, for example, in the form of a computer program including a plurality of instructions executable by one or more processors. The computer-readable storage medium may be non-transitory.

At block 302, process 300 involves receiving data from a user device. The received data may include location data and/or vital sign information data. In some embodiments, the service provider may periodically receive location data from the user device. In some cases, location data may be received directly from the user device (e.g., obtained via a GPS installed in the user device). In some embodiments, location data related to a user device may be received from cellular towers in communication with the user device. For example, the location of the user device may be determined using cellular tower triangulation. Vital sign data may include any indication of a user's health status, and may include, by way of non-limiting example, heartrate, body temperature, breathing rate, perspiration level, or any other suitable health-related information. In some embodiments, the health status information for a particular user may be obtained from a separate sensor device in communication with the user device. At block 304, the service provider stores the received data in a data store. Additionally, the service provider may retrieve historic location data for a user and store that data for use in future analysis.

At block 306, process 300 involves determining a likelihood that the user associated with the user device is infected based on the received data. In some embodiments, this may involve comparing the received vital sign data to historic vital sign data stored in association with that user. For example, the service provider may determine that the user's core body temperature has been slowly increasing over the last couple of days over historic body temperatures for that user. The service provider may then determine based on this information that the user likely is experiencing a fever. The service provider may further determine a fever is one symptom for a current disease for which there is an outbreak. Based on this, the service provider may calculate a likelihood that the user has the disease based on a body temperature determined for the user. In some embodiments, this may involve the use of one or more machine learning techniques. In some embodiments, a likelihood may be represented as a percentage or probability that the user is infected with a particular disease.

At decision block 308, process 300 involves determining whether the user is likely infected with a disease. To do this, the likelihood of infection determined at 306 may be compared to a threshold value. If the likelihood of infection is greater than the threshold value, the user may be determined to be infected. In some embodiments, the user may be categorized based on data received about the user. For example, the user may be categorized as category 1-4 based on his or her likelihood of being infected. In this example, category 1 may include individuals that have been confirmed to be infected (e.g., via self-reporting or via reporting by an external source). Category 2 may include individuals that are suspected of being infected (e.g., one or more biometric data received from the individual corresponds to biometric data expected of an infected individual). Category 3 may include individuals that have been determined to be in close proximity to individuals that are Category 1 or Category 2, but do not yet have symptoms of their own (i.e., biometric data for the individual does not match biometric data expected of an infected individual). Category 4 may include individuals that do not fit into categories 1-3. In such embodiments, a user's likelihood of infection may be determined based on his or her categorization. For example, if the user falls within categories 1-3, then that user may be considered to be likely infected. In contrast, if the user falls within category 4, then that user may be considered to be unlikely infected.

If the user is determined to not be infected (“no” from the decision block 308), then the process 300 may involve continuing to monitor the user by returning to block 302 and continuing to receive new data as indicated in block 310.

If the user is determined to be infected (“yes” from the decision block 308), then process 300 involves retrieving historic location data for the user as indicated in block 312. In some embodiments, the location data retrieved for the user may include location data that corresponds to a predetermined period of time from the current date/time. For example, upon determining that the user has just contracted a particular disease, the service provider may retrieve location data associated with the user for a period of time that corresponds to an incubation time for the disease. Additionally, the service provider may retrieve historic location data for a number of other users determined to be infected. In some embodiments, a number of previously-generated heat maps may be retrieved from a data store in order to be updated to include the new information.

At block 314, process 300 involves generating (or updating) a number of heat maps that include aggregated location data from the determined infected users. In some cases, location data for each user may be used to generate an area that includes some predetermined radius around the user's location. For example, the service provider, when generating a heat map, may assign a first value to an area within six feet of a likely-infected user's location, a second value to an area greater than six feet away but less than 12 feet away from the user's location, and a third value to an area between 12 and 20 feet from a likely infected user's location, in which the first value is higher than the second value, and the second value is higher than the third value. Overlapping values for multiple users may then be added. Each section of the heat map is then assigned a category (or color) based on the determined value for that section. Generated heat maps are then stored in a data store by the service provider.

At block 316, process 300 involves providing a notification to a third party. In some embodiments, the process involves providing the generated heat map to the third party. For example, upon generating one or more heat maps, the service provider may make accessible or otherwise provide those heat maps to an emergency service provider for a region depicted in the heat map. Note that such a disclosure would not include personal details for any of the likely-infected users.

At block 318, process 300 may involve performing additional processing in relation to the generated heat map. For example, in some embodiments, the service provider may, upon generating or updating a heat map, identify any other users that were, or are currently, located within a hotspot for the heat map. This may involve identifying an area corresponding to the hotspot at a particular point in time and determining whether a historic, or current, location stored in association with a user falls within that area. In some embodiments, the service provider may assign a risk value to various situations. For example, a user may be determined to be at a higher risk if the user is determined to have walked through, and spent a substantial amount of time within, a hotspot. In contrast, the user may be determined to be at much lower risk if the user is determined to have quickly driven through a hotspot. If the risk value is greater than some predetermined threshold value, then the service provider may provide a notification to that user as depicted at block 316, indicating that the user may have been infected. In some embodiments, a list of additional users that have come into contact with a likely-infected user may be stored by the service provider. Such a list may be provided to medical personnel for the purposes of contact tracing.

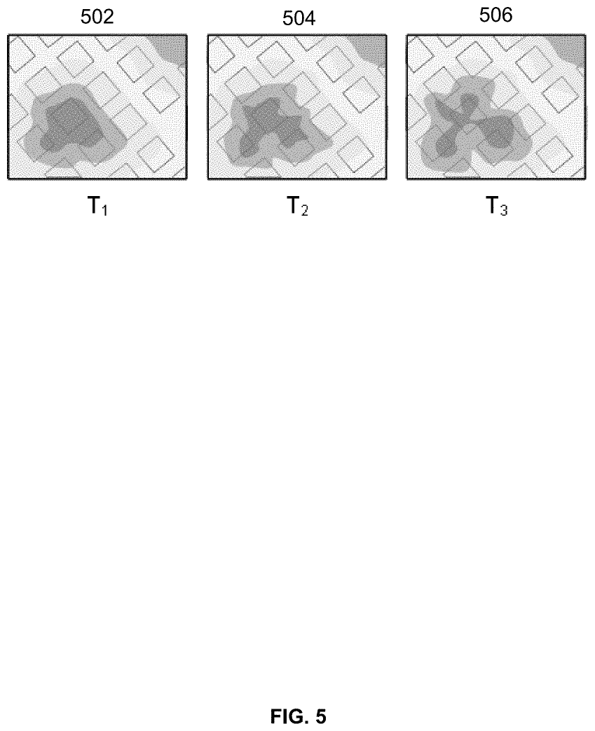

For the purposes of this disclosure, it should be noted that a heat map may be associated with a particular point in time, such that a number of heat maps may be stored for a particular geographical region, each representing a status of the geographical region at a particular point in time. For example, by way of illustration, consider three exemplary heat maps as depicted in FIG. 5 for a single geographic region.

FIG. 4 depicts a flow diagram illustrating an exemplary process for categorizing and tracking users in accordance with embodiments. The process 400 is illustrated as a logical flow diagram, each operation of which represents a sequence of operations that can be implemented in hardware, computer instructions, or a combination thereof

At 402 of the process 400, data associated with a user may be received. In some embodiments, such data is received from a user device operated by that user. Such data may include biometric data for the user as well as location data (both historical and current) for that user. In some embodiments, such data is received from one or more external sources, such as a government agency or health care administrator. Such data may include an indication of an infection status for the user and/or image data that includes an image identified as representing the user.

At 404 of the process 400, a user may be categorized based on the data received about that user. For example, a user may be associated with one of four categories based on the received data for that user.

In process 400, a user may be associated with a category 1 if that user is determined to be a known infected individual. A user may be a known infected individual if he or she is reported as being infected (e.g., self-reported or reported by a health care agency or other authoritative entity). In some embodiments, category 1 individuals may be monitored and reports may be generated and provided to external entities at 406 based on that monitoring. For example, the system may provide ongoing reports to a health care agency that includes biometric data for the user. In some cases, a first responder (e.g., emergency medical technician) may be contacted upon detecting a sudden change in the user's condition via the received biometric data. In some embodiments, a user device operated by a category 1 individual may provide an indication of any other user devices that come into proximity of that user device (e.g., via Bluetooth® or another suitable short-range wireless communication channel). This information may then be used to identify individuals to be classified as category 2 or category 3.

In process 400, a user may be associated with a category 2 if that user is determined to be a suspected infected individual. A user may be a suspected infected individual if his or her biometric data (e.g., as received from a user device) match or otherwise correspond to biometric data expected of an infected individual to a predetermined threshold degree. For example, if the biometric data received from the user is closer to matching biometric data expected of an infected individual as opposed to biometric data expected of an uninfected individual or a baseline (e.g., typical) biometric data for that user, then the user may be classified as a category 2 individual.

In process 400, a user may be associated with a category 3 if that user is determined to have come into substantial contact with an infected or suspected infected individual (e.g., category 1 and 2 individuals). Such a determination may be made based on a degree (e.g., a distance and amount of time) of contact between the individual and the infected or suspected infected individual. For example, the individual may have been determined to have come into contact with an infected or suspected infected individual within the last 14 days. In this example, the determination to categorize the individual as a category 3 individual may be made if that individual was within 6 feet of the infected individual for more than some threshold amount of time (e.g., one hour).

In some embodiments, if a user has been categorized as a category 3 individual, the process 400 may further comprise re-categorizing the user at 408 after a period of time. In these embodiments, such a recategorization may occur after some predetermined threshold amount of time has occurred since the individual was determined to come into contact with the infected or suspected infected individual. For example, at a point in time that is 14 days after a user classified as a category 3 individual has come into contact with an infected or suspected infected individual, the process may further comprise determining whether that user is exhibiting symptoms of infection (e.g., based on received biometric data). At this point, the user may be recategorized as either a category 2 (if the biometric data for the user matches biometric data expected of an infected individual) or a category 4 (if the biometric data for the user does not match biometric data expected of an infected individual).

In process 400, a user may be associated with a category 4 if that user does not fall into any of the other categories 1-3. For example, the user may be associated with a category 4 if the user's biometric data matches a baseline biometric data for that user or biometric data expected of an uninfected individual and the user has not come into contact with individuals classified as category 1-3.

In each of the cases in which the user is classified as belonging within categories 1-3, the process 400 may further comprise monitoring the user's biometric data and location at 410 in order to assess a current condition of that user as well as to identify other individuals that have come into contact with the user. In some embodiments, users may be re-categorized based on more recently received biometric data. For example, upon determining that the user's biometric data now more closely matches that of an uninfected individual or his or her baseline biometric data, the process may determine that the user has recovered from the infection and recategorize the user as a category 4 user. In another example, an indication may be received from an external source (e.g., a health care facility) that the user has recovered.

FIG. 5 depicts a number of example heat maps that may be generated in accordance with embodiments. In FIG. 5, an example heat map 502 is associated with a point in time T₁, example heat map 504 is associated with a point in time T₂, and example heat map 506 is associated with a point in time T₃. In some embodiments, the heat map associated with the latest point in time may be considered a current heat map.

In some embodiments, each of the heat maps 502, 504, and 506 may be updated as new information is received. For example, when an infected individual is newly identified, that infected individual's historical movements (e.g., locations and times at those locations) over a period of time may be determined based on his or her account information. In some cases, the period of time may correspond to a period of time over which the infected individual has been contagious. Once determined, each heat map relevant to the infected individual may be updated based on the historical movements for that infected individual. Particularly, density information included within each of the relevant (i.e., based on location and time) heat maps may be updated to reflect the presence of the infected individual.

In some embodiments, a likelihood of infection can be determined for a user using the generated heat maps. In order to do this, the user's historical movement information may be retrieved and compared to the relevant heat maps to identify the user's exposure to the infectious disease. A risk of infection may be calculated using an algorithm that takes as input the user's proximity to other infected individuals (e.g., how close the user got to the infected individual(s)), the length of time that the user spent in proximity, the infectiousness of the disease (e.g., how easily the disease is spread), whether the user is utilizing personal protective equipment (PPE), or any other suitable factor. In some embodiments, the algorithm may take into account the user's proximity to one or more hotspots. In order to assess whether a user has entered within a hotspot, that user's location data at any particular time may be compared to various heat maps for the particular times.

In some embodiments, a heat map may include information about determined categories of individuals. For example, a heat map may include multiple layers, where each of those multiple layers is generated for a particular category of individual (e.g., categories 1-4 as described with respect to FIG. 4 above). In these embodiments, a user's likelihood of infection may be calculated based on an aggregate of the information about each of the categories in the heat map.

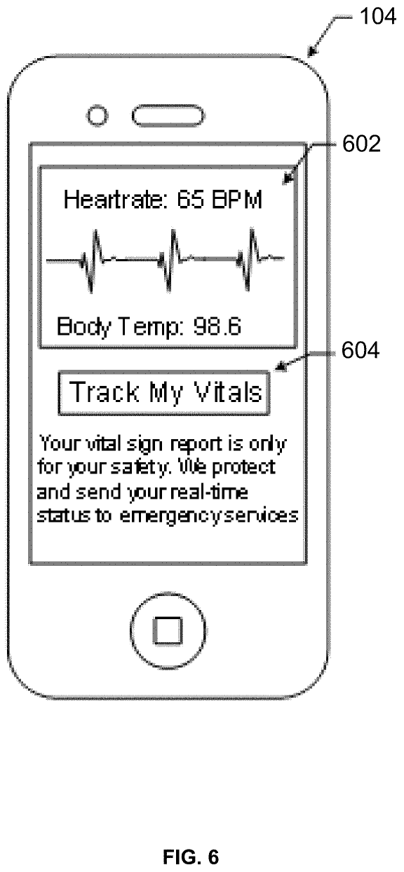

As noted elsewhere, the system described herein may be in communication with one or more user devices operated by various users (e.g., user devices 104 and 116 of FIG. 1). In some embodiments, the user devices may include one or more software application configured to enable various features as described herein. For example, the one or more software application may be an infection detection application 216 as described with respect to FIG. 2 above. Such applications may present information to a user via a graphical user interface (GUI). Some exemplary GUIs are presented below with respect to FIG. 6-8 that demonstrate various features of the recited system.

FIG. 6 depicts an exemplary GUI in which a user may be presented with vital sign information in accordance with some embodiments. As depicted, the user device 104 may receive vital sign information 602. In some embodiments, at least a portion of vital sign information 604 may be collected from one or more sensors installed upon the user device. In some embodiments, at least a portion of the vital sign information 104 may be received from an external sensor device (e.g., sensor device 110) in short-range communication with the user device. In these embodiments, the sensor device may be configured to collect any suitable biometric information about the user. Such biometric information may include a user's heart rate, body temperature, sweat production, or any other suitable indication of metrics associated with the user's biological function.

In some embodiments, the user may be asked for consent to provide the received vital sign information 602 to a service provider to be used in accordance with the embodiments described herein. In some embodiments, consent may be indicated via a button 604 or other GUI component.

In some embodiments, a likelihood of the user having been infected with an infectious disease may be determined based on the received vital sign information. In some cases, this may comprise providing the vital sign information 602 to a machine learning model that has been trained to correlate vital sign information to a probability of infection. In such cases, the machine learning model may be trained using vital sign information from both individuals that are known to have been infected and individuals that are known to have not been infected as inputs and an infected/uninfected status as outputs. In some embodiments, the likelihood of the user having been infected may be determined by the user device 104 using the trained machine learning model. In other embodiments, the likelihood of the user having been infected may be determined by a service provider platform that is remote to the user device 104 as described herein (e.g., service provider platform 200 as described in FIG. 2 above).

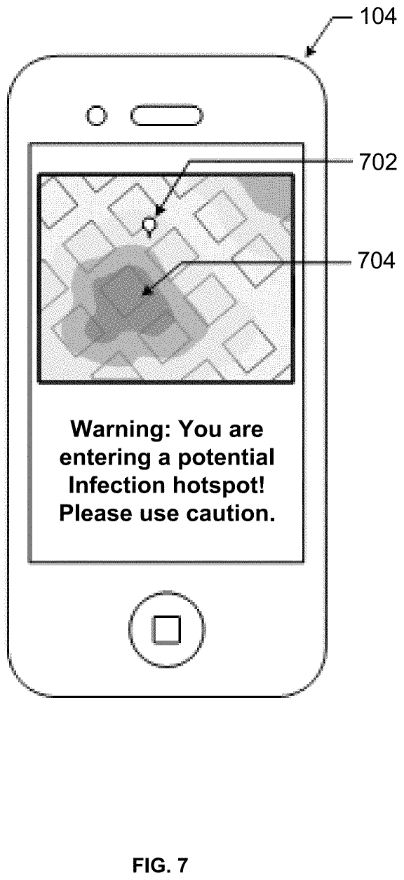

FIG. 7 depicts an exemplary GUI in which a user may be presented with a notification that he or she may be entering an infected hotspot in accordance with the embodiments described herein. In some embodiments, the service provider may make such a determination based on the user's current location. In some embodiments, the user may be provided his or her current location 702 as well as an indication of the relevant hotspot 704.

In some embodiments, a user's location may be tracked and compared against one or more heat maps for a region within which the user is located. In these embodiments, the user's location may be compared against a current heat map, which represents the latest generated heat map for the region within which the user is located. In some embodiments, a heat maps for a region may be generated or updated periodically (e.g., every 30 minutes, etc.) in order to ensure that a current heat map is current to less than a threshold amount of time.

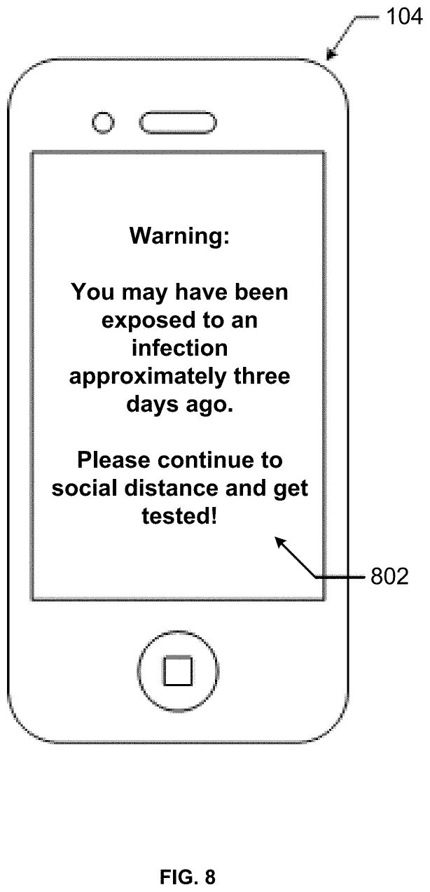

FIG. 8 depicts an exemplary GUI in which a user may be presented with a notification 802 that he or she may have been exposed to a disease in accordance with the embodiments described herein. In some embodiments, the service provider may make such a determination based on the user's historical locations in comparison with various hotspots identified in generated heat maps. In some embodiments, the user may be provided an indication of the time or date that the user may have been exposed.

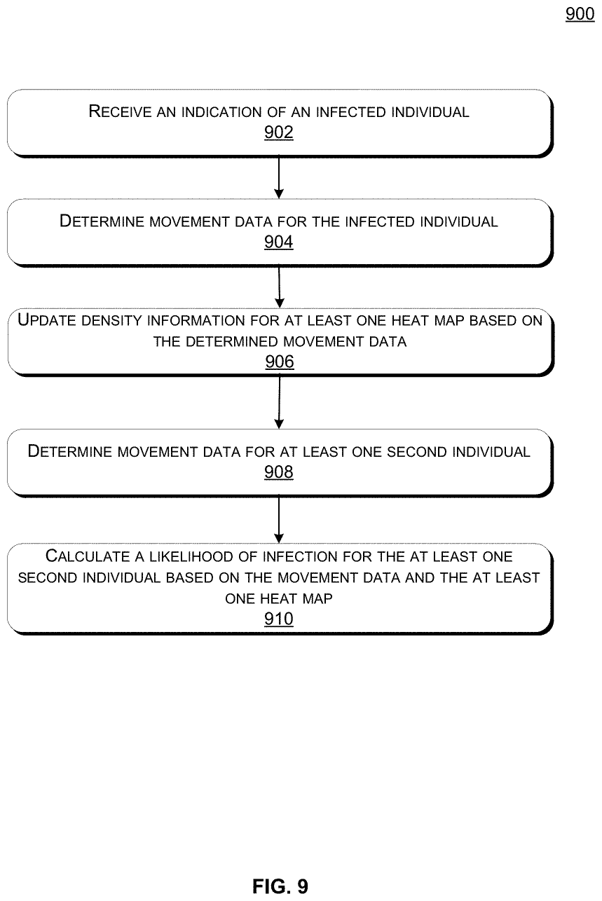

FIG. 9 depicts a block diagram showing an example process flow for generating one or more heat maps in accordance with some embodiments. In accordance with embodiments, the process 900 may be performed by components within a system 100 as discussed with respect to FIG. 1 above. For example, the process 900 may be performed by a service provider computer 108 in communication with a number of user devices 104 and/or 116.

At 902, the process 900 comprises receiving an indication of an infected individual. In some embodiments, the indication of the infected individual is received from one or more external sources. Such external sources may comprise at least one of a hospital, a clinic, or a laboratory. In some embodiments, the indication of the infected individual is received from a user device associated with the infected individual. For example, the user may report his or her infection of a disease via a mobile application installed upon the user's mobile device. In some embodiments, the indication of the infected individual is received upon determining that a likelihood of the infected individual being infected is greater than a threshold likelihood value. For example, biometric data may be received in relation to an individual and a likelihood of that individual may then be calculated based on that biometric data . In some cases, such biometric data is received from a sensor device in communication with a user device of the infected individual.

At 904, the process 900 comprises determining movement data for the infected individual. In some embodiments, the movement data for the infected individual is determined for an amount of time that corresponds to an incubation period for the infectious disease.

At 906, the process 900 comprises updating density information for at least one heat map based on the determined movement data. In at least some of these embodiments, the density information comprises an indication of a number of infected individuals per area. Such density information may be specific to a point in time, such that the density information included within the one or more heat maps varies at different points in time.

At 908, the process 900 comprises determining movement data for at least one second individual. In some embodiments, the movement data for the at least one second individual is determined for an amount of time that corresponds to an incubation period for the infectious disease.

At 910, the process 900 comprises calculating a likelihood of infection for the at least one second individual based on the movement data and the at least one heat map. In some embodiments, the likelihood of infection for the at least one second individual is calculated by a machine learning model. In at least some of these embodiments, the likelihood of infection for the at least one second individual is calculated based on the updated density information, a length of exposure, and a contagiousness of the infectious disease.

In some embodiments, the process 900 may further comprise determining one or more hotspots of infection within a geographical region. In some cases, the one or more hotspots of infection are determined based on the density information for the hotspot being greater than a threshold density. In some embodiments, the likelihood of infection for the at least one second individual is calculated based on at least a proximity of the at least one second individual to the one or more hotspots of infection.

In some embodiments, the process 900 may further comprise comparing the determined likelihood of infection for the at least one second individual to a threshold likelihood value. In some embodiments, upon determining that the likelihood of infection for the at least one second individual is greater than a threshold likelihood value, the process 800 may further comprise providing a notification to a user device associated with the at least one second individual.

### CONCLUSION

Although the subject matter has been described in language specific to features and methodological acts, it is to be understood that the subject matter defined in the appended claims is not necessarily limited to the specific features or acts described herein. Rather, the specific features and acts are disclosed as exemplary forms of implementing the claims.

What is claimed is:

1. A method comprising:
receiving, at a service provider in relation to an infectious disease, an indication of an infected individual;
determining, by the service provider, first movement data for the infected individual;
updating, by the service provider, density information included within one or more heat maps based on the first movement data for the infected individual;
determining, by the service provider, second movement data for at least one second individual; and
calculating, by the service provider, a likelihood of infection for the at least one second individual based on the second movement data and the updated one or more heat maps.

2. The method of claim 1, wherein the indication of the infected individual is received from one or more external sources.

3. The method of claim 2, wherein the one or more external sources comprises at least one of a hospital, clinic, or laboratory.

4. The method of claim 1, wherein the indication of the infected individual is received from a user device associated with the infected individual.

5. The method of claim 1, wherein the indication of the infected individual is received upon determining that a likelihood of the infected individual being infected is greater than a threshold likelihood value.

6. The method of claim 5, wherein the likelihood of the infected individual being infected is determined based on biometric data received in relation to the infected individual.

7. The method of claim 6, wherein the biometric data is received from a sensor device in communication with a user device of the infected individual.

8. A computing device comprising:
a processor; and
a memory including instructions that, when executed with the processor, cause the computing device to, at least:
receive, in relation to an infectious disease, an indication of an infected individual;
determine a first movement data for the infected individual;
update density information included within one or more heat maps based on the first movement data for the infected individual;
determine a second movement data for at least one second individual; and
calculate a likelihood of infection for the at least one second individual based on the second movement data and the updated one or more heat maps.

9. The computing device of claim 8, wherein the density information comprises an indication of a number of infected individuals per area.

10. The computing device of claim 8, wherein the instructions further cause the computing device to determine one or more hotspots of infection within a geographical region.

11. The computing device of claim 10, wherein the one or more hotspots of infection are determined based on the density information for the hotspot being greater than a threshold density.

12. The computing device of claim 10, wherein the likelihood of infection for the at least one second individual is calculated based on at least a proximity of the at least one second individual to the one or more hotspots of infection.

13. The computing device of claim 8, wherein the likelihood of infection for the at least one second individual is calculated by a machine learning model.

14. The computing device of claim 8, wherein the likelihood of infection for the at least one second individual is calculated based on the updated density information, a length of exposure, and a contagiousness of the infectious disease.

15. The computing device of claim 8, wherein the density information included within the one or more heat maps is specific to a point in time.

16. The computing device of claim 15, wherein the density information included within the one or more heat maps varies at different points in time.

17. The computing device of claim 8, wherein the first movement data for the infected individual is determined for an amount of time that corresponds to an incubation period for the infectious disease.

18. A non-transitory computer-readable media collectively storing computer-executable instructions that upon execution cause one or more computing devices to collectively perform acts comprising:
receiving, in relation to an infectious disease, an indication of an infected individual;
determining a first movement data for the infected individual;
updating density information included within one or more heat maps based on the first movement data for the infected individual;
determining a second movement data for at least one second individual; and
calculating a likelihood of infection for the at least one second individual based on the second movement data and the updated one or more heat maps.

19. The computer-readable media of claim 18, wherein the movement data for the at least one second individual is determined for an amount of time that corresponds to an incubation period for the infectious disease.

20. The computer-readable media of claim 18, wherein the instructions further cause one or more computing devices to, upon determining that the likelihood of infection for the at least one second individual is greater than a threshold likelihood value, provide a notification to a user device associated with the at least one second individual.
